(19) United States
(12) Patent Application Publication
(10) Pub. No.: US 2023/0293259 A1
(43) Pub. Date: Sep. 21, 2023

(54) SURGICAL SYSTEMS, METHODS, AND DEVICES EMPLOYING AUGMENTED REALITY (AR) GRAPHICAL GUIDANCE

(71) Applicant: Depuy Synthes Products, Inc., Raynham, MA (US)

(72) Inventor: Roman Lomeli, Plymouth, MA (US)

(21) Appl. No.: 18/122,802

(22) Filed: Mar. 17, 2023

Related U.S. Application Data
(60) Provisional application No. 63/321,618, filed on Mar. 18, 2022.

(51) Int. Cl. A61B 90/00; A61B 34/20; A61B 34/10

(52) U.S. Cl. CPC A61B 90/37; A61B 34/20; A61B 34/10; A61B 2034/107; A61B 2090/365; A61B 2034/105; A61B 17/1659

(57) ABSTRACT

Described are methods and systems for computer aided surgery (CAS) comprising an augmented reality (AR) system configured to display augmented reality information, a position tracking system configured to track positions of objects, an instrument coupled to a navigational tracker detectable by the position tracking system, and a controller configured to determine a position of the instrument, based on the determined position, display augmented reality information using the AR system, the augmented reality information comprising a representation of a relationship between at least a distal end of the instrument and tissue of the patient, and if the instrument moves to a second position, updating the representation. Examples of representations include a field of view of the instrument, a working volume of the instrument, a travel path of the instrument, adjacent patient structures, and other objects generally obscured by patient tissue.

### CROSS REFERENCE TO RELATED APPLICATION

This application claims priority to U.S. Provisional Application Ser. No. 63/321,618, filed Mar. 18, 2022, the contents of which are hereby incorporated by reference in its entirety.

### BACKGROUND

Many surgical procedures require large amounts of information for planning and/or undertaking the procedure. One way to manage this is to improve the way information is presented to a user, e.g., a surgeon.

Augmented Reality (AR) provides an overlay of virtual information on or adjacent to a “real-world” object visually perceived by a user, usually through an AR device such as a headset, Google Glass, etc. An AR device is configured to display information, such as pictures, video, text, warnings, models, simulations, etc., while not obscuring the user's view of the real-world objects in her proximity.

However, the information displayed may be selectable, pertinent, and customizable. For example, it would be beneficial to provide information that helps a surgeon visualize items that can't be directly perceived. Furthermore, specific use cases may present challenges that can be at least ameliorated by properly configured AR systems.

Accordingly, there is a need for improved systems, methods, and devices to employ AR that can improve patient outcome and surgical efficiency.

### SUMMARY

Described are methods and systems for computer aided surgery (CAS) comprising an augmented reality (AR) system configured to display augmented reality information, a position tracking system configured to track positions of objects, an instrument coupled to a navigational tracker detectable by the position tracking system, and a controller configured to determine a position of the instrument, based on the determined position, display augmented reality information using the AR system, the augmented reality information comprising a representation of a relationship between at least a distal end of the instrument and a tissue of a patient, and if the instrument moves to a second position, updating the representation. Examples of representations include a field of view of the instrument, a working volume of the instrument, a travel path of the instrument, adjacent patient structures, and other objects generally obscured by patient tissue.

### BRIEF DESCRIPTION OF THE DRAWINGS

FIG. 1 depicts a system for Augmented Reality (AR) in a surgical setting.

FIG. 2A depicts a schematic of an instrument with a navigational tracker.

FIG. 2B depicts a schematic of an instrument with a navigational tracker according to another embodiment.

FIG. 3 depicts a schematic of an AR display with camera orientation visualization.

FIG. 4 depicts a schematic of an AR display with a projected working volume.

FIG. 5 depicts graphs of maps of travel paths of an instrument.

FIG. 6 depicts a schematic of a determined skin surface position by tracking with an instrument.

### DETAILED DESCRIPTION

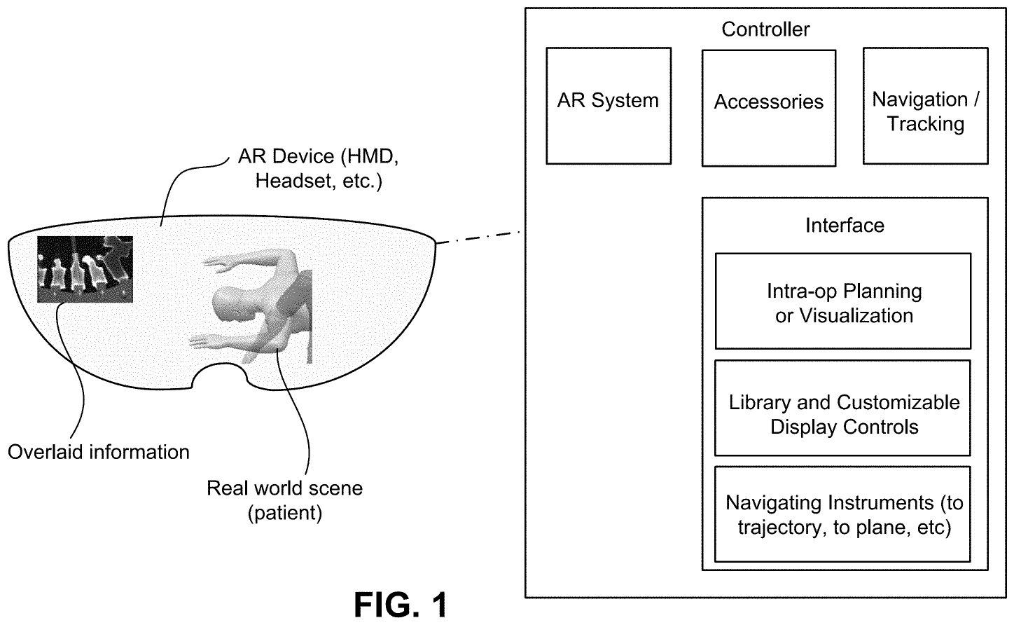

FIG. 1 depicts a system for Augmented Reality (AR) in a surgical setting. A user (e.g., surgeon) views a patient or other real-world object (instruments, operating room (OR) features, etc.) while receiving an overlay of virtual information from the controller. The information may be stored information or streamed information. Examples of information include pictures, video, text, warnings, models, simulations, etc. The information displayed may be selectable, pertinent, and customizable. For example, intra-op planning may greatly benefit from AR systems, provided it does not negatively impact workflow. Moreover, implementations for managing various information types (such as within a library) is challenging in a surgical setting. Furthermore, specific use cases, such as position-finding of instruments relative to a patient, may present challenges that may be at least ameliorated by properly configured AR systems.

Methods and implementations are provided to assist a surgeon to perform intra-operative (intra-op) visualization and/or planning from an AR headset, with minimal impact to their workflow. AR provides control to the surgeon, for example, for orthopedic procedures. Example applications include knee surgery (e.g., total knee arthroplasty (TKA) or uni-compartmental knee arthroplasty (UKA)), hip surgery (e.g., hip arthroplasty), shoulder surgery, spine surgery, and other orthopedic surgeries. In some embodiments, the system may enhance what the surgeon may see and help the surgeon visualize what they can't see. The display may include 3D CT model overlayed on native anatomy or suspended above the patient. The display may include virtual targets on the anatomy and information related to the instrument relative to the target. The display may include simultaneous high resolution video feeds (blood flow, nerves, etc.). There may be a contextual content to a current step in the workflow (e.g., a bone overlay may not be needed at the same time as seeing nerve or blood flow).

Provided is an AR system that has a user interface (e.g., with a controller) and a display, such as is typically associated with a headset. As will be described, navigation/tracking may be provided. In some embodiments, this application is directed to computer aided surgery (CAS) comprising an augmented reality (AR) system configured to display augmented reality information, a position tracking system configured to track positions of objects, an instrument coupled to a navigational tracker detectable by the position tracking system, and a controller configured to determine a position of the instrument, based on the determined position, display augmented reality information using the AR system. The controller may be used to send and receive information to and from the AR system. The controller typically includes a power supply, AC/DC converters, control system interface circuits, and other components included in computer aided surgery. The controller is also configured to perform the systems and methods described herein.

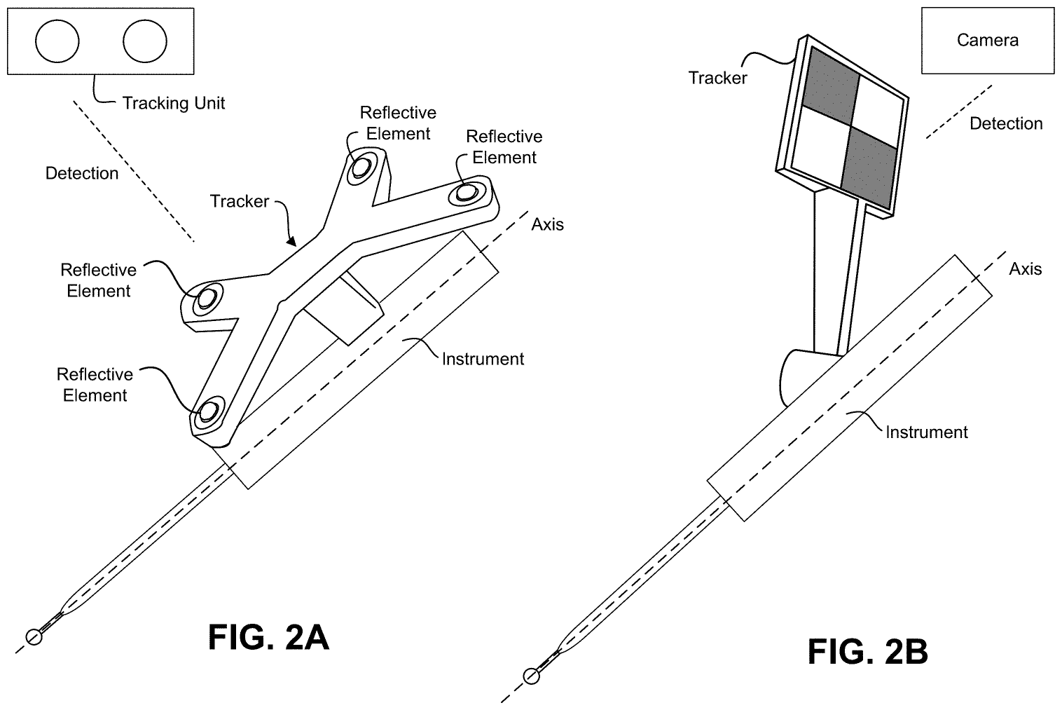

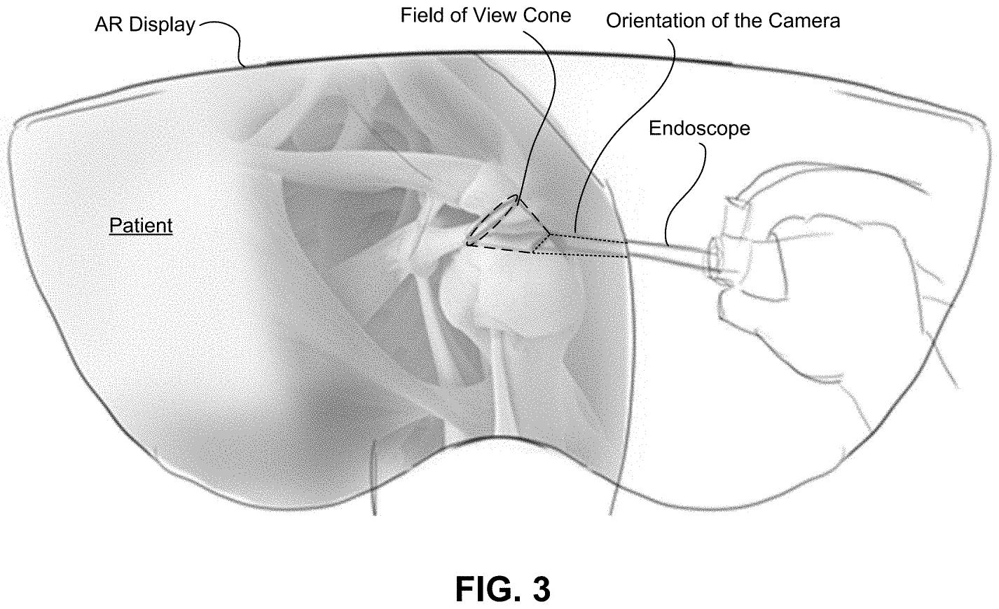

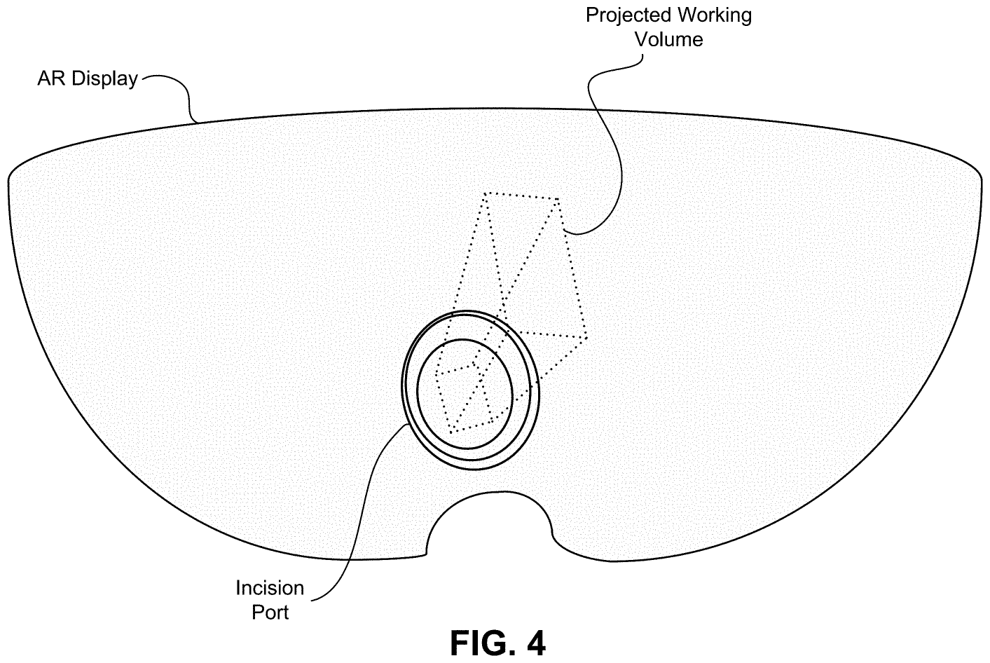

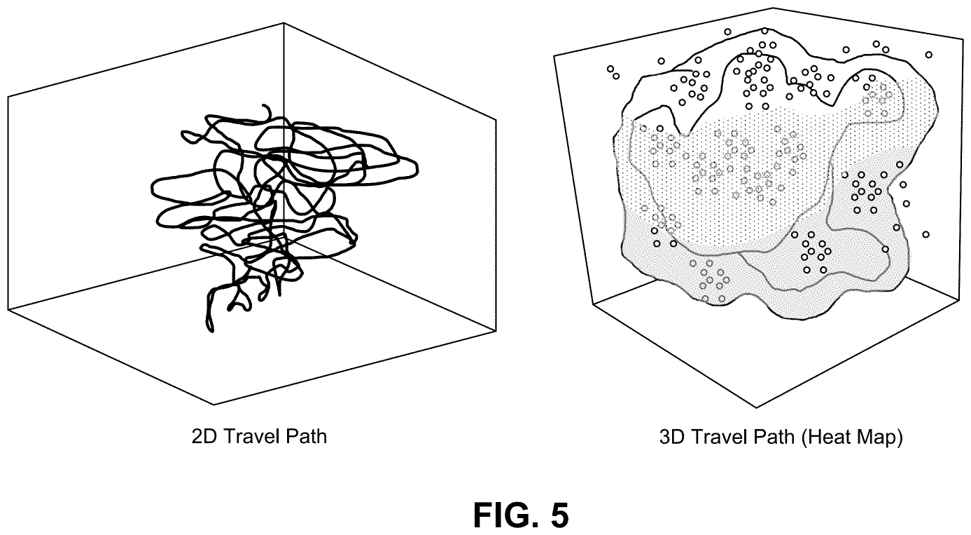

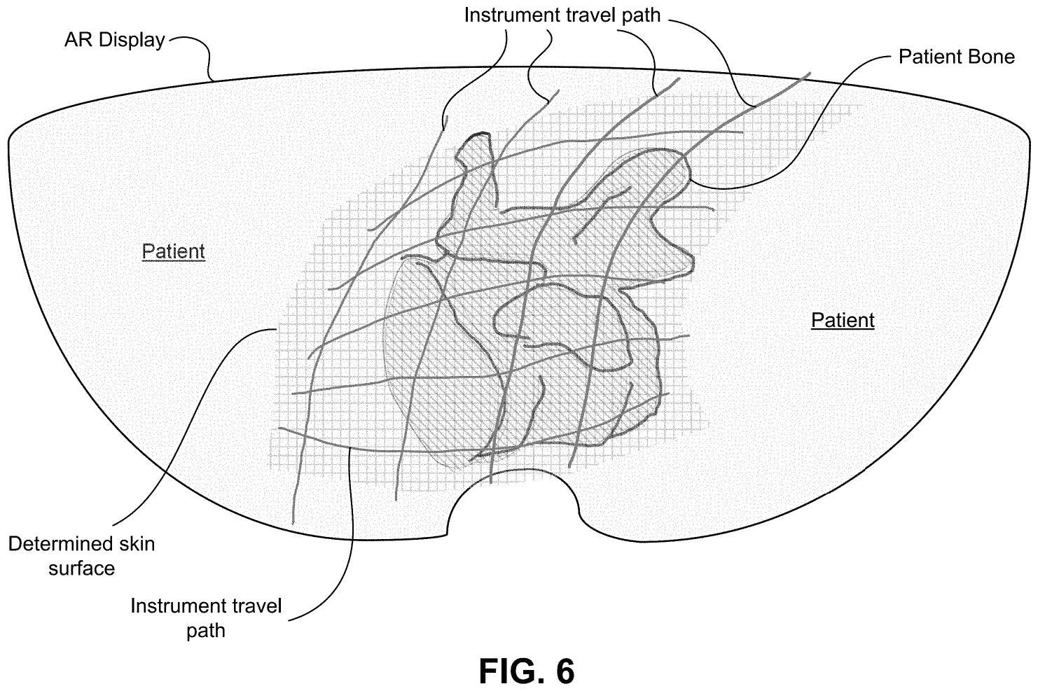

FIG. 2A depicts a schematic of an instrument with a navigational tracker. The instrument may be a camera (such as an endoscope (FIG. 3)). The instrument may be an instrument to be used within a working volume (FIG. 4). The instrument may be a scraper (such as described with respect to FIG. 5). The instrument may be a blunt instrument to help map an anatomical feature of a patient (FIG. 6). The instrument may be a cutting instrument (e.g., such as a shaver, a rongeur, or an energy device (such as, for example, a radiofrequency ablation device)). The system may have a plurality of navigational features (e.g., trackers) to determine a position (e.g., location and orientation in a three-dimensional space).

A tracker comprising a navigation array including a plurality of markers in a unique constellation or geometric arrangement may be provided. For example, optical navigation or tracking systems may utilize stereoscopic sensors (of a tracking unit) to detect light emitting diodes (LEDs) or infra-red (IR) light reflected or emitted from one or more optical markers affixed to the array. For example, when the markers are reflective elements, as illustrated, once detected by stereoscopic sensors, the relative arrangement of the elements in the sensors' field of view, in combination with the known geometric arrangement of the elements, may allow the system to determine a three-dimensional position of the array, and hence the instrument. Other examples of tracking systems in include ultrasonic sensors, radio-frequency identification (RFID) sensors or other radio frequency (RF) tracking systems, electromagnetic interference (EMI) tracking systems, visual systems including for example chest markers, Aruco markers, etc.

The tracker may reveal a position of the instrument. Stated differently, the tracker may help provide complete positioning information (e.g., of the instrument), which may be used by the controller. An additional tracker (not depicted) may be attached to a patient, thus allowing the position of the instrument to be relative to the patient. The controller may determine (or be informed of) a position of a patient anatomy. The additional tracker may be present elsewhere in an operating theater, e.g., such as coupled to a surgical table. One non-limiting example of a surgical table is a cervical traction frame.

The additional tracker may assist with tracking an anatomy of interest, for example, a shoulder, a pelvis, a femur, a tibia, or a pedicle of the spine. A patient coordinate system may be defined to refer to the position of the patient with respect to the instrument. The navigation system (e.g., such as the tracking unit) may track the tracker(s) for purposes of displaying their relative positions and orientations to the surgeon and, in some cases, for displaying virtual information associated with the patient's anatomy.

The tracking unit may include one or more navigation system cameras that may capture a position of the markers (e.g., reflective elements as depicted). The navigation cameras may be stereoscopic. The relative pose or three-dimensional position (e.g., location and orientation) of the tracker may be tracked and shared with the controller. The tracking unit may measure the relative motions between any and all trackers in real time. This information may thus identify a position of the instrument to which the tracker is coupled in three-dimensional space given the known and precise relationship between the tracker and the instrument. For example, the controller may be configured to identify a 3D position of a portion of the instrument, such as a tip.

A computer assisted surgical system may comprise the above-described navigational tracker with a plurality of optical tracking elements, an optical tracking unit, and a controller adapted to utilize a predetermined fixed geometric relationship between the tracking elements and detected positions of the tracking elements to determine a position (e.g., three-dimensional) of the instrument.

FIG. 2B depicts a schematic of an instrument with a navigational tracker according to another embodiment. The instrument may be a camera (such as an endoscope (FIG. 3)). The instrument may be an instrument to be used within a working volume (FIG. 4). The instrument may be a scraper (such as described with respect to FIG. 5). The instrument may be a blunt instrument to help map an anatomical feature of a patient (FIG. 6). The instrument may be a cutting instrument (e.g., such as a shaver, a rongeur, or an energy device (such as, for example, a radiofrequency ablation device)).

In this embodiment, the tracker is one that is detectable by a camera of the headset (AR system), or alternatively by a separate camera mounted to the headset, or alternatively by a camera separate and located remotely from the headset. For example, the tracker may be a chest marker used for camera pose estimation. The tracker may reveal a position of the instrument (e.g., the tracker may help provide complete positioning information of the instrument which may be used by the controller). The camera may capture a position of the tracker. The relative pose or three-dimensional position (e.g., location and orientation) of the tracker may be tracked and shared with the controller. This information may thus identify a position of the instrument to which the tracker is coupled in three-dimensional space given the known and precise relationship between the tracker and the instrument. For example, the controller may be configured to identify a 3D position of a portion of the instrument, such as a tip.

An additional tracker (not depicted) may be attached to a patient, thus allowing the position of the instrument to be relative to the patient. The controller may determine (or be informed of) a position of a patient anatomy (a shoulder, a femur, a tibia, or a pedicle of the spine). The additional tracker may be present elsewhere in an operating theater, e.g., such as coupled to a surgical table. One non-limiting example of a surgical table is a cervical traction frame. The additional tracker may assist with tracking an anatomy of interest. A patient coordinate system may be defined to refer to the position of the patient with respect to the instrument. The camera may track the tracker(s) for purposes of displaying their relative positions and orientations to the surgeon and, in some cases, for displaying virtual information associated with the patient's anatomy.

A computer assisted surgical system may comprise the above-described camera, a chest tracker, and a controller adapted to utilize a predetermined fixed pattern of the tracker to determine a position of the instrument.

Regardless of the detection system employed above, the controller may determine a position of the instrument (for example, a distal end of the instrument (e.g., with respect to the surgeon)). The controller may also determine a position of the patient (for example, a patient anatomy). The controller may be configured to cause the AR system to display, such as on the headset, augmented reality information comprising a representation of a relationship between a distal end of the instrument and tissue of the patient. The controller may be configured to, if the instrument moves to a second position, cause the AR system to display an updated representation (e.g. updating the representation). Examples of representations include a field of view of the instrument, a working volume of the instrument, a travel path of the instrument, adjacent patient structures, and other objects generally obscured by patient tissue, as will now be described in greater detail.

The following examples may be used with navigational tracker(s) detected by a camera of the AR system or detected by stereoscopic cameras (e.g., of a tracking unit).

FIG. 3 depicts a schematic of an AR display with camera orientation visualization. It is understood that the tracker(s), tracking unit/camera, and controller are providing the augmented reality information. As the surgeon inserts a camera (e.g., an endoscope) into a patient, it can be appreciated that the tip of the instrument is obscured by patient tissue. The controller may be configured to cause the AR system to display (such as in an overlay view or X-ray view) a representation that is a field of view of the instrument extending from the distal end of the instrument. The representation may include an orientation of the camera and a projection of the field of view cone, e.g., to aid the surgeon in orienting to the patient's anatomy (e.g., in MIS or endoscopic procedures). The representation of the field of view of the instrument may be a three dimensional representation of a field of view of an endoscopic camera superimposed over the tissue of the patient, thereby providing a predicted indication of the view of the tissue of the patient from the endoscopic camera.

The controller may be configured to cause the AR system to display a feature of the patient. For example, the surface of the bone within the cone may be highlighted. This will aid the user to orient to the anatomy and make sure they are focused on the area of interest (this also has application in orthopedic and general surgery). The controller may be further configured to determine a virtual view simulating a view of the tissue of the patient from a point of view of the endoscopic camera, and cause the AR system to display the virtual view simulating the view of the tissue of the patient from the point of view of the endoscopic camera. Other virtual anatomical features may be overlaid as part of the display.

FIG. 4 depicts a schematic of an AR display with a projected working volume. It is understood that the tracker(s), tracking unit/camera, and controller are providing the augmented reality information. As a surgeon uses an instrument in an incision in a patient, it can be appreciated that the tip of the instrument is obscured by patient tissue. The controller may be configured to receive planning information regarding a position on a patient where the instrument is to be used, and a working volume of the instrument based on the planning information. The controller may be configured to cause the AR system to display a representation comprising a working volume of the instrument based on the planning information.

In one such example, the planning information comprises information regarding a vertebral body. In this example, the representation comprises a predicted working volume of a portion of the instrument that corresponds to the distal end of the instrument being disposed at the vertebral body. The instrument may be a scraper and the predicted working volume may correspond to the distal end of the scraper being disposed in an intervertebral disc space between two vertebral bodies. In some embodiments, the predicted working volume may be illustrated as a cube (a projected working volume). When the surgeon needs to work within a defined space of the anatomy, a working volume may be projected outside of the patient representing the extent of the instruments that will be used within the space. For example, the working volume may be maintained as long as a handle of the instrument does not leave the displayed projected working volume.

Alternatively, a projected working volume may allow the user to visually reference and determine if they have reached all areas of the planned working space. In a spine, this may be used for discectomy. A projected working volume may be displayed, or because the different instruments may have a different geometry, the projected working volume may be specific to a geometry of each instrument. An upper and lower limit to the projected working volume may represent the extent the user may desire to operate the instrument. For discectomy, the lower limit is important to avoid breaching anteriorly, multiple lower limits may be displayed to show the user is approaching a critical structure. This may be displayed through virtual planes, or virtual blocks shown with different colors, transparencies, or other visually distinct methods.

In another example, the system allows virtual geofencing, such as virtually painting surgical plans onto the patient. The surgeon may indicate where instruments are planned to access (e.g., displayed in green) and where the user does not want instruments to access (e.g., displayed in red). The latter may remind the user to avoid points or planes that represent structures such as nerves, soft tissue, etc. The virtual geofencing may be stored in the controller such that when a tool is about to enter a no-go area, the user is warned with an alert (color changes, audible, and the like). Or if the surgeon is using a robotically assisted system the geofencing can be implemented with haptics or the like. In a robotic or power tool system, geofencing may also be tied to power modulation (turning it on/off or adjusting the speed) and an energy device may also be modulated in the same way, as well as combined devices like an RF shaver, that has energy and mechanical cutting.

In another example, the controller may be configured to display a working volume of a planned interbody in a user's view (e.g., to ensure that enough bony removal has been conducted) or during an annulotomy step. Working volumes can also be used to show, for example, where a planned implant will sit (like spine & trauma plates), bone that should be removed (osteotomies, osteophytes), or a planned tissue resection for cancerous or damaged tissue.

FIG. 5 depicts graphs of maps of travel paths of an instrument. For example, when a surgeon is working within a defined space (discectomy) with a tracked instrument, a series of consecutive positions of the instrument tip (e.g., a travel path) may be determined and stored (e.g., by the controller) over time. The controller may be configured to display a visual representation of where the instrument has been inside the space (2D travel path, 3D travel path, 3D heat map, etc.). The controller may be configured to display this information (for example, superimposed onto an axial view of the endplate), which may give the surgeon a better understanding of how much disc prep they've completed. For example, a 3D representation of the volume of a disc space may give the surgeon a better understanding of how well each endplate has been prepared. Alternatively, the controller may be configured to display a virtual disc representation that may disappear as the instrument moves over a given area, for example, the removal may be tied to the number of passes or time.

In some embodiments, the representation is a travel path of the distal end of the instrument over time. The travel path may be displayed with an indication of portions of the travel path that are more heavily traveled and more lightly traveled. The travel path may be displayed to indicate areas that are predicted as requiring more traversing of the distal end of the instrument. For example, the instrument may be a scraper, the tissue of the patient may be an intervertebral disc space between two vertebral bodies, and an indication may be provided for an area of disc space predicted as requiring more scraping before insertion of an intervertebral implant.

In yet another example, the controller is further configured to use the travel path to determine an envelope of excised tissue from the patient. This envelope might correspond to the amount of intervertebral disc space removed from a patient. Thus, a surgeon might be able to view the envelope to determine if a particular intervertebral implant would fit. Further, the system might make such determination and recommend to the surgeon whether to continue removing intervertebral disc. The system might further recommend an implant size for insertion

In some embodiments, the controller may be configured to use the travel path, for example of a ball tipped pointer (such illustrated in FIG. 2A or 2B), to map a skin surface (as part of determining a desired position of a future incision) on the patient.

FIG. 6 depicts a schematic of a determined skin surface position by mapping with an instrument. For example, multiple instrument travel paths may be traced across a patient's skin. For example, by painting the surface of the skin by running the tip of the instrument along the operative area (alternatively, the skin surface can be displayed as a perpendicular intersection to the tip of the instrument). The controller may be configured to determine a 3D position of the skin surface. The controller may be configured to use the travel path to determine a position of a surface of the patient's skin.

For example, a surgeon may map the skin so that she knows where to make a skin incision, an angle/approach for the incision, and/or an appropriate width of the incision. In some embodiments, the surgeon may traverse the patient's skin with the ball-tipped pointer and the system may measure positions on the skin using the tracked instrument before making an incision. An anatomy (e.g., bone) overlay size may be adjusted manually or by proximity of the tracked instrument (e.g., to focus in on a specific vertebra closest to the instrument). A surgeon may measure distances (e.g., such as between two selected positions on a bone overlay) using a projection from an instrument axis before making an incision. The bone may be displayed in an anatomy overlay on the patient. The surgeon may mark a starting position then move to a secondary position on the bony surface. As the user moves to the secondary position, a live dimension may be displayed or an extension line may be displayed. This dimension may be point to point or follow the contour of the bone. Optionally, a depth to bone may also be displayed. This may allow planning of an incision, an access trajectory, which port to use, or a length of instrument to use. This this information may also be used to select implant sizes (for example, surgical plates for spine and trauma). For example, the controller may (may also) be configured to use the travel path to determine an angle and a width of the scalpel incision. Optionally, a virtual incision may be displayed to guide a surgeon (e.g., during blunt dissection).

If the headset contains two integrated cameras, a 3D point cloud may be generated of the skin surface and then be utilized to create a surface level overlay of the exact skin incision point with dimensions applied.

In an example use case, for a percutaneous AR procedure without pre-operative planning, a surgeon may intra-operatively use the tracked instrument to plan the trajectory of a pedicle screw (e.g., define the trajectory for pedicle screw placement and save that trajectory, optionally also capturing the tip position of the tool at intersection with the skin). The controller may have determined a position of a surface of the patient's skin. With a defined skin surface, the system would be able to display percutaneous implant intersection points with the skin surface and the surgeon would be able to modify the plan to minimize incision size and number of incisions. In some embodiments, the system may display an incision point and width for the selected implant. This may increase the efficiency of the interoperative planning process and reduce the number of times that incisions need to be expanded later in the procedure.

The controller may be further configured to display a virtual implant and skin surface intersection. A user may continue to plan the other screws. Optionally, the system would allow the surgeon to either keep the planned visualizations on or turn them off (e.g., to improve visibility). Once all screws are planned, the system may display all implants and skin intersections. The surgeon may have the option to modify any of the implant positions or use the incision indications to make the incision in the correct position, along the correct access, and the correct length.

In some embodiments, the representation is a patient nerve adjacent to the instrument. For example, stored nerve positions may be overlaid. Alternatively, nerve scan or neuromonitoring results may be obtained to build a visual representation of where the neural anatomy lies under the tissue. Nerve positions may be displayed as a heat map on the patient. Alternatively, as the navigated instrument passes through the tissue, a color code may be applied to the region based upon the proximity of a nerve.

In some embodiments, the representation is a patient vascular structure adjacent to the instrument. For example, stored blood vessel positions may be overlaid. As the navigated instrument passes through the tissue, a color code may be applied to the region based upon the proximity of a blood vessel.

In some embodiments, the representation is a patient bone adjacent to the instrument. For example, overlays may be shown as a contour of the bone (e.g., rather than as a fully rendered 3D model). This may allow the surgeon to visualize the bone under the skin surface in a minimal form to reduce visual clutter and distraction. Different bone positions may be distinguished with transparency, color, outlines, etc. In another example, finding an endplate may be very difficult if it is collapsed and when the bone quality is poor. Improper targeting may lead to endplate damage, especially in patients with poor bone quality. The controller may be configured to display the endplates as planes in the user's view, and a user may target the disc space faster and with higher accuracy.

In a first embodiment, a computer aided surgery (CAS) system is provided. The CAS system may comprise an augmented reality (AR) system configured to display augmented reality information; a position tracking system configured to track positions of objects; an instrument coupled to a navigational tracker detectable by the position tracking system; and a controller configured to: determine a position of the instrument; based on the determined position, display augmented reality information using the AR system, the augmented reality information comprising a representation of a relationship between at least a distal end of the instrument and a tissue of a patient; and if the instrument moves to a second position, updating the representation. In some embodiments, the navigational tracker is detected by a camera of the AR system. In some embodiments, the navigational tracker is detected by stereoscopic cameras.

In some embodiments, the controller is further configured to display a representation that is a field of view of the instrument extending from the distal end of the instrument. In some embodiments, the representation of the field of view of the instrument is a three dimensional representation of a field of view of an endoscopic camera superimposed over the tissue of the patient, thereby providing a predicted indication of the view of the tissue of the patient from the endoscopic camera. In some embodiments, the controller is further configured to: determine a virtual view simulating a view of the tissue of the patient from a point of view of the endoscopic camera; and cause the AR system to display the virtual view simulating the view of the tissue of the patient from the point of view of the endoscopic camera.

In some embodiments, the controller is further configured to receive planning information regarding a position on a patient where the instrument is to be used, and the representation comprises a working volume of the instrument based on the planning information. In some embodiments, the planning information comprises information of a vertebral body. In some embodiments, the controller is further configured to display a representation that comprises a predicted working volume of a portion of the instrument that corresponds to the distal end of the instrument being disposed at the vertebral body. In some embodiments, the instrument is a disc removal tool and the predicted working volume corresponds to the distal end of the tool being disposed in an intervertebral disc space between two vertebral bodies.

In some embodiments, the controller is further configured to display a representation that is a travel path of the distal end of the instrument over time. In some embodiments, the travel path provides an indication of portions of the travel path that are more heavily traveled and more lightly traveled. In some embodiments, the travel path indicates areas that are predicted as requiring more traversing of the distal end of the instrument. In some embodiments, the instrument is a scraper, the tissue of the patient is an intervertebral disc space between two vertebral bodies, and the indication is an area of disc space predicted as requiring more scraping before insertion of an intervertebral implant. In some embodiments, the controller is further configured to use the travel path to determine a position of a surface of the patient's skin. In some embodiments, the controller is further configured to use the travel path to determine a desired position of an incision on the patient. In some embodiments, the controller is further configured to use the travel path to determine an envelope of excised tissue from the patient.

In some embodiments, the controller is further configured to display a representation that is a patient nerve adjacent to the instrument.

In some embodiments, the controller is further configured to display a representation that is a patient vascular structure adjacent to the instrument.

In some embodiments, the controller is further configured to display a representation that is a patient bone adjacent to the instrument.

In a second embodiment, a method of computer aided surgery (CAS) is provided. The method may be a pre-operative planning method. The method comprises determining a position of an instrument, wherein the instrument is coupled to a navigational tracker detectable by a position tracking system; displaying, on an augmented reality (AR) system, augmented reality information comprising a representation of a relationship between at least a distal end of the instrument and a tissue of a patient; and updating the representation if the instrument moves to a second position.

In some embodiments, the controller is further configured to display a representation that is a field of view of the instrument, a working volume of the instrument, a travel path of the instrument, or an adjacent patient structure.

The embodiments of the present disclosure described above are intended to be merely examples; numerous variations and modifications within the scope of this disclosure. Accordingly, the disclosure is not to be limited by what has been particularly shown and described. All publications and references cited herein are expressly incorporated by reference in their entirety, except for any definitions, subject matter disclaimers or disavowals, and except to the extent that the incorporated material is inconsistent with the express disclosure herein, in which case the language in this disclosure controls.

1. A computer aided surgery (CAS) system, comprising:
an augmented reality (AR) system configured to display augmented reality information;
a position tracking system configured to track positions of objects;
an instrument coupled to a navigational tracker detectable by the position tracking system; and
a controller configured to:
determine a position of the instrument;
based on the determined position, display augmented reality information using the AR system, the augmented reality information comprising a representation of a relationship between at least a distal end of the instrument and a tissue of a patient; and
if the instrument moves to a second position, updating the representation.

2. The system of claim 1, wherein the representation is a field of view of the instrument extending from the distal end of the instrument.

3. The system of claim 2, wherein the representation of the field of view of the instrument is a three dimensional representation of a field of view of an endoscopic camera superimposed over the tissue of the patient, thereby providing a predicted indication of the view of the tissue of the patient from the endoscopic camera.

4. The system of claim 1, wherein the controller is further configured to:
determine a virtual view simulating a view of the tissue of the patient from a point of view of the endoscopic camera; and
cause the AR system to display the virtual view simulating the view of the tissue of the patient from the point of view of the endoscopic camera.

5. The system to claim 1, wherein the controller is further configured to receive planning information regarding a position on a patient where the instrument is to be used, and the representation comprises a working volume of the instrument based on the planning information.

6. The system of claim 5, wherein the planning information comprises information of a vertebral body.

7. The system of claim 6, wherein the representation comprises a predicted working volume of a portion of the instrument that corresponds to the distal end of the instrument being disposed at the vertebral body.

8. The system of claim 7, wherein the instrument is a disc removal tool and the predicted working volume corresponds to the distal end of the tool being disposed in an intervertebral disc space between two vertebral bodies.

9. The system of claim 1, wherein the representation is a travel path of the distal end of the instrument over time.

10. The system of claim 9, wherein the travel path provides an indication of portions of the travel path that are more heavily traveled and more lightly traveled.

11. The system of claim 9, wherein the travel path indicates areas that are predicted as requiring more traversing of the distal end of the instrument.

12. The system of claim 11, wherein the instrument is a scraper, the tissue of the patient is an intervertebral disc space between two vertebral bodies, and the indication is an area of disc space predicted as requiring more scraping before insertion of an intervertebral implant.

13. The system of claim 9, wherein the controller is further configured to use the travel path to determine a position of a surface of the patient's skin.

14. The system of claim 9, wherein the controller is further configured to use the travel path to determine a desired position of an incision on the patient.

15. The system of claim 9, wherein the controller is further configured to use the travel path to determine an envelope of excised tissue from the patient.

16. The system of claim 1, wherein the representation is a patient nerve adjacent to the instrument.

17. The system of claim 1, wherein the representation is a patient vascular structure adjacent to the instrument.

18. The system of claim 1, wherein the representation is a patient bone adjacent to the instrument.

19. A method of computer aided surgery (CAS), comprising:
determining a position of an instrument, wherein the instrument is coupled to a navigational tracker detectable by a position tracking system;
displaying, on an augmented reality (AR) system, augmented reality information comprising a representation of a relationship between at least a distal end of the instrument and a tissue of a patient; and
updating the representation if the instrument moves to a second position.

20. The method of claim 19, wherein the representation is a field of view of the instrument, a working volume of the instrument, a travel path of the instrument, or an adjacent patient structure.
